(19) United States
(12) Patent Application Publication
(10) Pub. No.: US 2008/0319031 A1
(43) Pub. Date: Dec. 25, 2008

(54) Novel Tyrosine Derivatives

(75) Inventors: Surendrakumar Satyanarayan Pandey, Sholinganallur (IN); Shiv Kumar Agarwal, New Delhi (IN); Gajendra Singh, Sholinganallur (IN); Santhanagopalan Chithra, Sholinganallur (IN); Sangmesh Badiger, Bangalore (IN); Bishwajit Nag, Union City, CA (US); Debendranath Dey, Union City, CA (US); Abhijeet Nag, Union City, CA (US); Partha Neogi, Union City, CA (US)

Correspondence Address: FOLEY AND LARDNER LLP;SUITE 500, 3000 K STREET NW, WASHINGTON, DC, 20007, US

(73) Assignee: Orchid Chemicals & Pharmaceuticals Limited

(21) Appl. No.: 11/886,460

(22) PCT Filed: Mar. 10, 2006
(86) PCT No.: PCT/IB2006/000525

§ 371 Date: May 21, 2008

(30) Foreign Application Priority Data
Mar. 18, 2005 (IN) 279/CHE/2005

(51) Int. Cl. A61K 31/428; A61P 3/00; A61P 29/00; A61P 37/00; A61P 35/00; A61P 3/04; C07D 209/32; C07D 277/68; A61K 31/404

(52) U.S. Cl. 514/367; 548/165; 548/486; 514/418

(57) ABSTRACT

The present invention relates to novel tyrosine derivatives of formula (I), their pharmaceutically acceptable salts and pharmaceutically acceptable compositions containing them. The present invention more particularly provides novel tyrosine derivatives of the general formula (I).

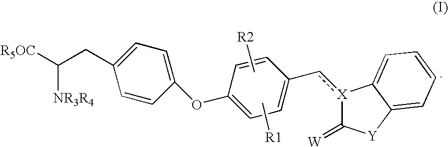

### FIELD OF THE INVENTION

The present invention relates to novel tyrosine derivatives of formula (I), their pharmaceutically acceptable salts and pharmaceutically acceptable compositions containing them. The present invention more particularly provides novel tyrosine derivatives of the general formula (I)

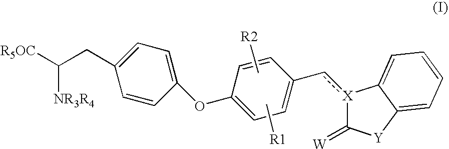

The present invention also relates to a process for the preparation of the above said novel compounds, their pharmaceutically acceptable salts and pharmaceutically acceptable compositions containing them.

The compounds of the present invention are effective in lowering blood glucose, serum insulin, free fatty acids, cholesterol and triglyceride levels and are useful in the treatment and/or prophylaxis of type II diabetes. The compounds of the present invention are effective in treatment of obesity, inflammation, autoimmune diseases such as multiple sclerosis and rheumatoid arthritis. Surprisingly, these compounds increase the leptin level and have no liver toxicity.

Furthermore, the compounds of the present invention are useful for the treatment of disorders associated with insulin resistance, such as polycystic ovary syndrome, as well as hyperlipidemia, coronary artery disease and peripheral vascular disease, and for the treatment of inflammation and immunological diseases, particularly those mediated by cytokines such as TNF-α, IL-1, IL-6, IL-1β and cyclooxygenase such as COX-2.

### BACKGROUND OF THE INVENTION

The causes of type I and II diabetes are not yet clear, although both genetics and environment seem to be the factors. Type I is an autonomic immune disease and patient must take insulin to survive. Type II diabetes is more common form, is metabolic disorder resulting from the body's inability to make a sufficient amount of insulin or to properly use the insulin that is produced. Insulin secretion and insulin resistance are considered the major defects, however, the precise genetic factors involved in the mechanism remain unknown.

Patients with diabetes usually have one or more of the following defects:
Less production of insulin by the pancreas;
Over secretion of glucose by the liver;
Independent of the glucose uptake by the skeletal muscles;
Defects in glucose transporters, desensitization of insulin receptors;
Defects in the metabolic breakdown of polysaccharides.

Other than the parenteral or subcutaneous administration of insulin, there are about 4 classes of oral hypoglycemic agents used i.e. sulfonylurea, biguanides, alpha glucosidase inhibitors and thiazolidinediones.

Each of the current agents available for use in treatment of diabetes has certain disadvantages. Accordingly, there is a continuing interest in the identification and development of new agents, which can be orally administered, for use in the treatment of diabetes.

The thiazolidinedione class listed above has gained more widespread use in recent years for treatment of type II diabetes, exhibiting particular usefulness as insulin sensitizers to combat “insulin resistance”, a condition in which the patient becomes less responsive to the effects of insulin. There is a continuing need for nontoxic, more widely effective insulin sensitizers. In our continuous efforts to explore new compounds having antidiabetic activity, we propose to synthesis new compounds containing oxindole and benzothiazolone systems.

Recent advances in scientific understanding of the mediators involved in acute and chronic inflammatory diseases and cancer have led to new strategies in the search for effective therapeutics. Traditional approaches include direct target intervention such as the use of specific antibodies, receptor antagonists, or enzyme inhibitors. Recent breakthroughs in the elucidation of regulatory mechanisms involved in the transcription and translation of a variety of mediators have led to increased interest in therapeutic approaches directed at the level of gene transcription.

As indicated above, the present invention is also concerned with treatment of immunological diseases or inflammation, notably such diseases as are mediated by cytokines or cyclooxygenase. The principal elements of the immune system are macrophages or antigen-presenting cells, T cells and B cells. The role of other immune cells such as NK cells, basophils, mast cells and dendritic cells are known, but their role in primary immunologic disorders is uncertain. Macrophages are important mediators of both inflammation and providing the necessary “help” for T cell stimulation and proliferation. Most importantly macrophages make IL 1, IL 12 and TNF-α all of which are potent pro-inflammatory molecules and also provide help for T cells. In addition, activation of macrophages results in the induction of enzymes, such as cyclooxygenase II (COX-2), inducible nitric oxide synthase (iNOS) and production of free radicals capable of damaging normal cells. Many factors activate macrophages, including bacterial products, superantigens and interferon gamma (IFN γ). It is believed that phosphotyrosine kinases (PTKs) and other undefined cellular kinases are involved in the activation process.

Cytokines are molecules secreted by immune cells that are important in mediating immune responses. Cytokine production may lead to the secretion of other cytokines, altered cellular function, cell division or differentiation. Inflammation is the body's normal response to injury or infection. However, in inflammatory diseases such as rheumatoid arthritis, pathologic inflammatory processes can lead to morbidity and mortality. The cytokine tumor necrosis factor-alpha (TNF-α) plays a central role in the inflammatory response and has been targeted as a point of intervention in inflammatory disease. TNF-α is a polypeptide hormone released by activated macrophages and other cells. At low concentrations, TNF-α participates in the protective inflammatory response by activating leukocytes and promoting their migration to extravascular sites of inflammation (Moser et al., J Clin Invest, 83:444-55,1989). At higher concentrations, TNF-α can act as a potent pyrogen and induce the production of other pro-inflammatory cytokines (Haworth et al., Eur J Immunol, 21:2575-79, 1991; Brennan et al., Lancet, 2:244-7, 1989). TNF-α also stimulates the synthesis of acute-phase proteins. In rheumatoid arthritis, a chronic and progressive inflammatory disease affecting about 1% of the adult U.S. population, TNF-α mediates the cytokine cascade that leads to joint damage and destruction (Arend et al., Arthritis Rheum, 38:151-60,1995). Inhibitors of TNF-α, including soluble TNF receptors (etanercept) (Goldenberg, Clin Ther, 21:75-87, 1999) and anti-TNF-α antibody (infliximab) (Luong et al., Ann Pharmacother, 34:743-60, 2000), have recently been approved by the U.S. Food and Drug Administration (FDA) as agents for the treatment of rheumatoid arthritis.

Elevated levels of TNF-α have also been implicated in many other disorders and disease conditions, including cachexia, septic shock syndrome, osteoarthritis, inflammatory bowel disease such as Crohn's disease and ulcerative colitis etc.

It can be seen that inhibitors of TNF-α are potentially useful in the treatment of a wide variety of diseases. Compounds that inhibit TNF-α have been described in several patents.

Excessive production of IL-6 is implicated in several disease states, it is highly desirable to develop compounds that inhibit IL-6 secretion. Compounds that inhibit IL-6 have been described in U.S. Pat. Nos. 6,004,813; 5,527,546 and 5,166,137.

The cytokine IL-1β also participates in the inflammatory response. It stimulates thymocyte proliferation, fibroblast growth factor activity, and the release of prostaglandin from synovial cells.

Elevated or unregulated levels of the cytolcine IL-1β have been associated with a number of inflammatory diseases and other disease states, including but not limited to adult respiratory distress syndrome, allergy, Alzheimer's disease etc.

Since overproduction of IL-1β is associated with numerous disease conditions, it is desirable to develop compounds that inhibit the production or activity of IL-1β.

It will be appreciated from the foregoing facts that, while there have been extensive prior efforts to provide compounds for inhibiting, for example, TNF-α, IL-1, IL-6, COX-2 or other agents considered responsible for immune response, inflammation or inflammatory diseases, e.g. arthritis, there still remains a need for new and improved compounds for effectively treating or inhibiting such diseases.

With an objective of providing compounds, which are effective for such treatments as well as for the treatment of, for example, insulin resistance, hyperlipidemia, obesity, inflammation, multiple sclerosis, arthritis, atherosclerosis, autoimmune disease and cancer.

Few prior art reference which disclose the closest compounds are given here:

i) US publication No. US 2004/0142991 discloses compounds of formula (I)

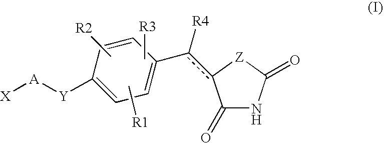

wherein - - - represents optional double bond; Y represents oxygen, sulfur or NR, wherein R represents hydrogen or alkyl; Z represents oxygen or sulfur; R₁, R₂, R₃ and R₄ may be same or different and independently represent hydrogen, halogen, hydroxy, nitro, cyano, formyl, amino, alkyl, alkoxy group; A represents a bond or substituted or unsubstituted aryl, heterocyclyl or heteroaryl ring; X represents amino acid or its derivatives

The compounds of this formula is shown in Example (1)

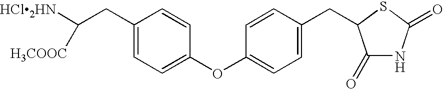

ii) International publication No. WO 93/00337 discloses chemical structure of (I) in the treatment of diabetes and which have useful pharmacological properties, producing an action on the intermediate metabolism and in particularly lowering of blood-sugar level;

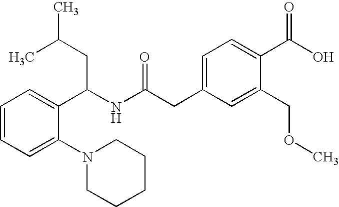

iii) U.S. Pat. No. 4,572,912 discloses compounds of formula (I) and a series of new thiazolidine derivatives, which likewise have the ability to lower blood lipid and blood sugar levels.

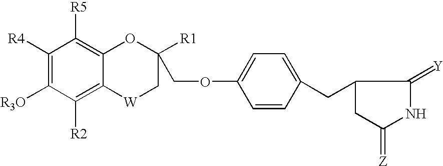

R1 and R2 are the same or different and each represents hydrogen or C1-C5 alkyl; R3 represents hydrogen, an acyl group, a (C1-C6 alkoxy) carbonyl group or an aralkyloxycarbonyl group; R4 and R5 are the same or different and each represents hydrogen, C1-C5 alkyl or C1-C5 alkoxy, or R4 and R5 together represent a C1 14 C4 allcylenedioxy group; n is 1, 2 or 3;

W represents the —CH2—, >CO or >CH—OR6 group (in which R6 represents any one of the atoms or groups defined for R3 and may be the same as or different from R3); and Y and Z are the same or different and each represents oxygen or imino.

IV) U.S. Pat. No. 4,687,777 discloses compounds of formula (I) and thiazolidinedione derivatives of the formula I and pharmacologically acceptable salts thereof are novel compounds, which exhibit in mammals blood sugar- and lipid-lowering activity, and are of value as a therapeutic agent for treatment of diabetes and hyperlipemia.

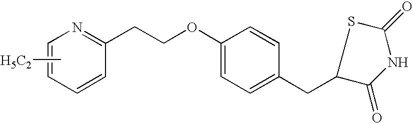

### OBJECTIVE OF THE INVENTION

With an objective to develop novel compounds for lowering blood glucose, free fatty acids, cholesterol and triglyceride levels in type II diabetes and to treat autoimmune diseases such as multiple sclerosis and rheumatoid arthritis, we focused our research to develop new compounds effective in the treatment of the above mentioned diseases. Effort in this direction has led to compounds having general formula (I).

The main objective of the present invention is therefore, to provide novel tyrosine derivatives, their pharmaceutically acceptable salts and pharmaceutical compositions containing them.

Another objective of the present invention is to provide novel tyrosine derivatives, their pharmaceutically acceptable salts and pharmaceutical compositions containing them or their mixtures that are useful for treatment of disorders associated with insulin resistance, such as polycystic ovary syndrome, as well as hyperlipidemia, coronary artery disease and peripheral vascular disease, and for the treatment of inflammation and immunological diseases, particularly those mediated by cytokines such as TNF-α, IL-1, IL-6, IL-1β and cyclooxygenase such as COX-2.

Another objective of the present invention is to provide novel tyrosine derivatives, their pharmaceutically acceptable salts and pharmaceutical compositions containing them or their mixtures having enhanced activities, without toxic effect or with reduced toxic effect.

Yet another objective of the present invention is to provide a process for the preparation of novel tyrosine derivatives of formula (I), their pharmaceutically acceptable salts and pharmaceutical compositions containing them or their mixtures.

### SUMMARY OF THE INVENTION

The present invention, relates to novel tyrosine derivatives of formula (I)

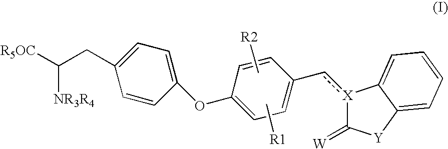

their pharmaceutically acceptable salts and pharmaceutical compositions containing them, wherein - - - represents an optional bond; W represents O or S; X represents C, CH or N; Y represents NR₆, S or O, wherein R₆ represents hydrogen, substituted or unsubstituted alkyl, —CH₂COOR, or aryl, or counter ion; wherein R represents H or alkyl group; R₁, R₂, may be same or different and independently represent hydrogen, halogen, hydroxy, nitro, cyano, formyl, amino, alkyl, haloalkyl, alkoxy group; R₃ and R₄ may be same or different and independently represent H, alkyl, COR₇, where R₇ represents H, substituted or unsubstituted groups selected from alkyl, haloakyl, aryl, alkenyloxy, aryloxy, alkoxy or arylalkoxy; R₅ represents —OR₈ where R₈ represents hydrogen, substituted or unsubstituted groups selected from alkyl, alkenyl, aryl, aralkyl, heteroaryl, or a counter ion, NR₉R₁₀, where R₉ and R₁₀ may be same or different and independently represent H, substituted or unsubstituted groups selected from alkyl, alkenyl, aryl.

### DETAILED DESCRIPTION OF THE INVENTION

In an embodiment of the present invention, the groups represented by R₁, R₂ are selected from hydrogen, halogen such as fluorine, chlorine, bromine or iodine; hydroxy, nitro, cyano, formyl, amino, linear or branched, substituted or unsubstituted (C₁-C₄) alkyl group such as methyl, ethyl, propyl, isopropyl, n-butyl, isobutyl, t-butyl and the like; haloalkyl groups selected from alkyl group substituted by one, two, three or four halogen atoms such as chloromethyl, chloroethyl, trifluoromethyl, trifluoroethyl, dichloromethyl, dichloroethyl and the like; substituted or unsubstituted (C₁-C₄)alkoxy group such as methoxy, ethoxy, propoxy, butoxy and the like.

Suitable groups represented by R₃ and R₄ may be same or different and independently represent H, COR₇; where R₇ represents H, substituted or unsubstituted groups selected from alkyl substituted or unsubstituted linear or branched C₁-C₄ alkyl group such as methyl, ethyl, n-propyl, isopropyl, n-butyl, isobutyl, t-butyl and the like, haloalkyl such as chloromethyl, chloroethyl, trifluoromethyl, trifluoroethyl, dichloromethyl, dichloroethyl and the like, aryl such as phenyl, naphthyl and the like, the aryl group may be substituted, alkoxy such as methoxy, ethoxy, propoxy n-butoxy, isobutoxy, t-butoxy and the like or aralkoxy, alkenyloxy, aryloxy.

Suitable groups represented by R₅ represent OR₈, NR₉R₁₀.

Suitable groups represented by R₆ are selected from hydrogen, substituted or unsubstituted alkyl, alkenyl, CH₂COOR, or aryl, or counter ion; wherein R represents H or alkyl group;

Suitable groups represented by R₈ are selected from hydrogen, substituted or unsubstituted linear or branched C₁-C₄ alkyl group such as methyl, ethyl, n-propyl, isopropyl, n-butyl, isobutyl, t-butyl; aryl such as phenyl; aralkyl group such as benzyl; counter ion selected from alkali metal like Li, Na, and K; alkaline earth metal like Ca and Mg; salts of different bases such as ammonium or substituted ammonium salts.

Suitable groups represented by R₉ and R₁₀ are selected from hydrogen, substituted or unsubstituted linear or branched C₁-C₄ alkyl group such as methyl, ethyl, n-propyl, isopropyl, n-butyl, isobutyl, t-butyl and the like; aryl such as phenyl;

Pharmaceutically acceptable salts forming part of this invention include base addition salts such as alkali metal salts like Li, Na, and K salts, alkaline earth metal salts like Ca and Mg salts, salts of organic bases such as lysine, arginine, guanidine, diethanolamine, choline and the like, ammonium or substituted ammonium salts. Salts may include acid addition salts which are sulphates, nitrates, phosphates, perchlorates, borates, hydrohalides, acetates, tartrates, maleates, citrates, succinates, palmoates, methanesulphonates, benzoates, salicylates, hydroxynaphthoates, benzenesulfonates, ascorbates, glycerophosphates, ketoglutarates and the like. Pharmaceutically acceptable solvates may be hydrates or comprising other solvents of crystallization such as alcohols.

Representative compounds according to the present invention include:
Methyl-2-amino-3-(4-{4-[(2-oxo-1,2-dihydro-3H-indol-3-ylidene)methyl]phenoxy}phenyl) propanoate hydrochloride;
Methyl-2-amino-3-(4-{4-[(2-oxo-2,3-dihydro-1H-indol-3-yl)methyl]phenoxy}phenyl) propanoate hydrochloride;
Methyl-2-amino-3-(4-{4-[(2-oxo-1,2-dihydro-3H-indol-3-ylidene)methyl]phenoxy}phenyl) propanoate hydrochloride;
Methyl-2-amino-3-(4-{3-chloro-4-[(2-oxo-1,2-dihydro-3H-indol-3-ylidene)methyl]phenoxy}phenyl) propanoate hydrochloride;
Methyl-2-amino-3-(4-{2-chloro-4-[(2-oxo-1,2-dihydro-3H-indol-3-ylidene) methyl]phenoxy}phenyl)propanoate hydrochloride;
Methyl-2-amino-3-(4-{2-fluoro-4-[(2-oxo-1,2-dihydro-3H-indol-3-ylidene) methyl]phenoxy}phenyl) propanoate hydrochloride;
Methyl-2-amino-3-(4-{2-trifluoromethyl-4-[(2-oxo-1,2-dihydro-3H-indol-3-ylidene)methyl]phenoxy}phenyl) propanoate hydrochloride;
Methyl-2-amino-3-(4-{3-trifluoromethyl-4-[(2-oxo-1,2-dihydro-3H-indol-3-ylidene)methyl]phenoxy}phenyl) propanoate hydrochloride;
Methyl-2-amino-3-(4-{2-methoxy-4-[(2-oxo-1,2-dihydro-3H-indol-3-ylidene)methyl]phenoxy}phenyl) propanoate hydrochloride;
Methyl-2-amino-3-(4-{4-[(2-oxo-2,3-dihydro-1H-indol-3-yl)methyl]phenoxy}phenyl) propanoate hydrochloride;
Methyl-2-amino-3-(4-{2-trifluoromethyl-4-[(2-oxo-2,3-dihydro-1H-indol-3-yl)methyl]phenoxy}phenyl) propanoate hydrochloride;
Methyl-2-amino-3-(4-{3-trifluoromethyl-4-[(2-oxo-2,3-dihydro-1H-indol-3-yl)methyl]phenoxy}phenyl) propanoate hydrochloride;
Methyl-2-amino-3-(4-{3-fluoro-4-[(2-oxo-2,3-dihydro-1H-indol-3-yl)methyl]phenoxy}phenyl) propanoate hydrochloride;
Methyl-2-amino-3-(4-{2-fluoro-4-[(2-oxo-2,3-dihydro-1H-indol-3-yl)methyl]phenoxy}phenyl) propanoate hydrochloride;
Methyl-2-amino-3-(4-{3-chloro-4-[(2-oxo-2,3-dihydro-1H-indol-3-yl)methyl]phenoxy}phenyl) propanoate hydrochloride;
Methyl-2-amino-3-(4-{2-methoxy-4-[(2-oxo-2,3-dihydro-1H-indol-3-yl)methyl]phenoxy}phenyl) propanoate hydrochloride;
Methyl-2-amino-3-(4-{2-chloro-4-[(2-oxo-2,3-dihydro-1H-indol-3-yl)methyl]phenoxy}phenyl) propanoate hydrochloride;
2-amino-3-{4-[4-(2-oxo-1,2-dihydroindol-3-ylidenemethyl)-phenoxy]-phenyl}-propionic acid hydochloride;
2-amino-3-{4-[4-(2-oxobenzothiazol-3-ylmethyl)-phenoxy]-phenyl}-propionic acid methyl ester hydrochloride;
2-amino-N,N-dimethyl-3-{4-[4-(2-oxo-1,2-dihydroindol-3-ylidenemethyl)-phenoxy]-phenyl}-propionamide hydrochloride;
2-amino-3-{4-[4-(2-oxo-1,2-dihydroindol-3-ylidenemethyl)-phenoxy]-phenyl}-propionamide hydrochloride.

Preferred salts for the list of compounds above are hydrochloride, hydrobromide, sodium, potassium or magnesium.

According to another feature of the present invention, there is provided a process for the preparation of compounds of formula (I), wherein - - - represents an optional bond and all other symbols are as defined earlier, as shown in scheme-I

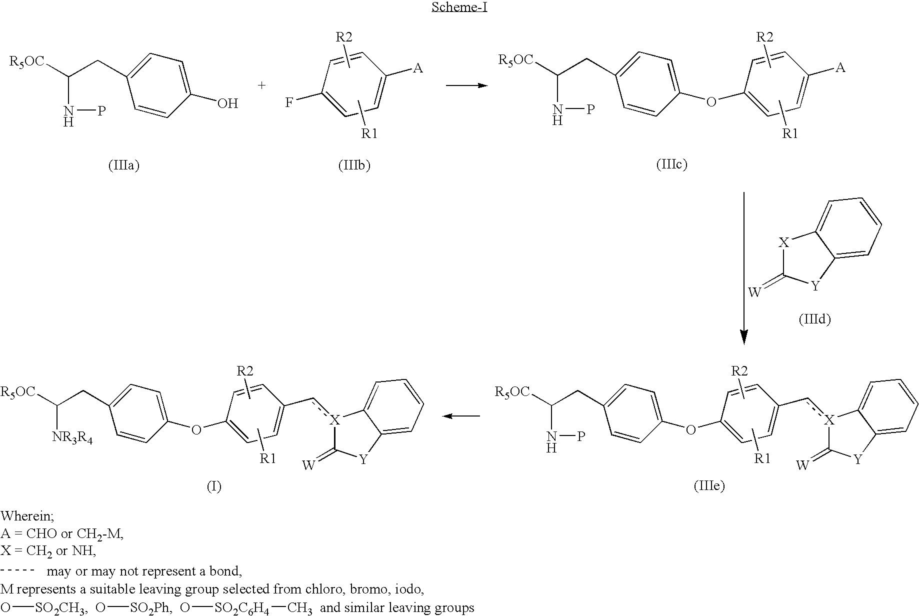

The reaction of compound of formula (IIIa) with the compound of formula (IIIb) produce a compound of formula (IIIc) in the presence of solvents such as THF, DMF, DMSO, DME and the like or mixtures of solvents may be used. The reaction may be carried out in an inert atmosphere. The reaction may be effected in the presence of a base such as K₂CO₃, Na₂CO₃, NaH or mixtures thereof. The reaction temperature may range from 20° C. to 150° C., preferably at a temperature in the range of 30° C. to 100° C. The duration of the reaction may range from 1 to 24 hours, preferably from 2 to 12 hours. The reaction of the compound of the general formula (IIIc) with a compound of formula (IIId) may be carried out at 100 to 180° C. in the presence of base and in the presence of a solvent such as toluene, methoxyethanol or mixtures thereof to yield a compound of formula (IIIe). The reaction temperature may range from 100° C. to 180° C., when the reaction is carried out neat in the presence of suitable catalyst such as piperidine, benzoic acid piperidinium acetate or benzoate, sodium acetate or mixtures of catalysts may also be employed. Piperidine can be used in the presence of solvent. The water produced in the reaction may be removed by using Dean Stark water separator or by using water-absorbing agents like molecular sieves.

The deprotection of formula (IIIe) to yield compound of formula (I) may be carried out using acids such as HCl, sulfuric acid, acetic acid , trifluoroacetic acid in the presence of solvents such as DCM, ethyl acetate, water and the like or mixture thereof at a temperature in the range of −10° C. to 50° C.

In another embodiment of the present invention, there is provided a process for the preparation of compounds of formula (I), by reducing the penultimate step of formula (I) wherein - - - represents bond. The reduction step is not required when - - - represent no bond and all other symbols are as defined earlier. The reduction may be carried out in the presence of gaseous hydrogen and a catalyst such as Pd/C, Rh/C, Pt/C, Raney Nickel, and the like. Mixtures of catalysts may be used. The reaction may be conducted in the presence of solvents such as methanol, dichloromethane, dioxane, acetic acid, ethyl acetate and the like. Mixtures of solvents may be used. A pressure between atmospheric pressure to 15 Kg/cm² may be employed. The catalyst may be 5-10% Pd/C and the amount of catalyst used may range from 50-300% w/w.

The protecting group P used in the invention are conventional protecting groups such as t-butoxy carbonyl (t-Boc), trityl, trifluoroacetyl, benzyloxy, benzyloxy carbonyl (Cbz) and the like. Deprotection can be done by conventional methods. The invention is explained in detail in the examples given below which are provided by way of illustration only and therefore should not be construed to limit the scope of the invention.

### EXAMPLE 1

Methyl-2-amino-3-(4-{4-[(2-oxo-1,2-dihydro-3H-indol-3-ylidene) methyl]phenoxy}phenyl) propanoate hydrochloride.

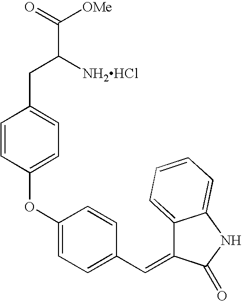

### Step I

Preparation of methyl 2-[(tert-butoxycarbonyl)amino]-3-[4-(4-formylphenoxy)phenyl]propanoate

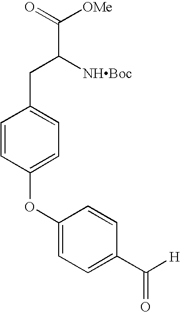

To the suspension of sodium hydride (0.81 gm, 33.8 mmol) in dry DMF (20 ml), under nitrogen charged BOC-tyr-OMe solution (10 gm, 33.8 mmol, dissolved in 20 ml DMF), in 30 minutes at 30° C. Green colored reaction mixture was obtained stirred for 15 minutes. p-Fluorobenzaldehyde solution (4.2 gm, 33.8 mmol, 5 ml DMF) was added to reaction mixture at 30° C. in 5 minutes. The reaction mixture was warm to 80° C. in 45 minutes. The colour of reaction mixture changed to brown after 2 hrs. The TLC shows absence of BOC-Tyr-OMe. The solvent was distilled under high vacuum. The thick reaction mass obtained was quenched with saturated ammonium chloride solution. The reaction mixture was extracted with ethyl acetate dried over sodium sulfate and concentrated. Finally the crude product was purified by column chromatography to yield the title compound. yield: 6.23 gm, ¹H NMR (CDCl₃, 400 MHz): 1.42 (s, 9H), 3.05 (m, 2H), 3.74 (s, 3H), 4.61 (q, 1H), 5.05 (d, 1H), 7.05 (m, 4H), 7.26 (d, 2H), 7.85 (d, 2H), 9.92 (s, 1H), m/z^{M+1} 400.2.

Step II

Preparation of Methyl-2[(tert-butoxycarbonyl)amino]-3-(4-{4-[(2-oxo-1,2-dihydro-3H-indol-3-ylidene)methyl]phenoxy}phenyl) propanoate

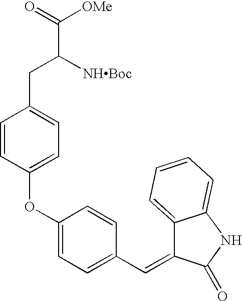

A solution of methyl 2-[(tert-butoxycarbonyl)amino]-3-[4-(4-formylphenoxy)phenyl] propanoate (2.0 gm, 5.0 mmol), 2-oxo-indole (0.8 gm, 6.0 mmol), benzoic acid (0.091 gm, 0.75 mmol) and piperidine (0.055 gm, 0.65 mmol) in toluene (50 ml) was refluxed with stirring at 145°-155° C. with continuous removal of water using dean stark apparatus for 3 hr. The reaction mixture was allowed to attain room temperature and concentrated. The crude product thus obtained was purified by column chromatography. Yield 1.8 gm; m/z^{M+1} 515.4.

Step III

Preparation of Methyl-2-amino-3-(4-{4-[(2-oxo-1,2-dihydro-3H-indol-3-ylidene)methyl]phenoxy}phenyl) propanoate hydrochloride.

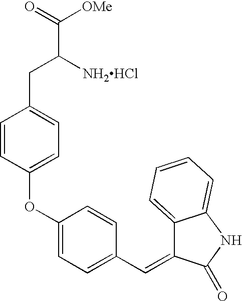

Compound from step II (0.8 gm, 1.56 mmol) was dissolved in CH₂Cl₂ (20 ml) and cooled to 0-5° C. Dry hydrogen chloride gas was bubbled through this solution for 20 min. After completion of the reaction the bubbling was discontinued and the reaction mixture was stirred at room temperature for 1 hour. The excess hydrochloric acid was degassed and the solvent was removed. The residual solid was triturated with ethyl acetate, filtered and dried to yield the title compound. (0.6 gm ); ¹H NMR (DMSO-d₆, 400 MHz) δ ppm: 3.0 (d, 2H), 3.6 (s, 3H), 4.1 (t, 1H), 6.8 (d, 2H), 7.0 (s, 1H), 7.1 (t, 4H), 7.3 (t,2H), 7.5 (d,2H), 7.7 (d,2H), 8.5 (bs,2H), 10.6 (s,1H); m/z^{M+1} 415.

### EXAMPLE 2

Synthesis of Methyl-2-amino-3-(4-{4-[(2-oxo-2,3-dihydro-1H-indol-3-yl)methyl]phenoxy}phenyl) propanoate hydrochloride.

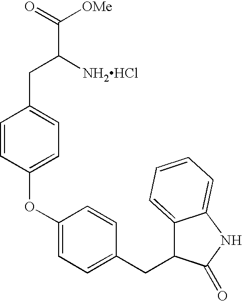

To the solution of Methyl-2-amino-3-(4-{4-[(2-oxo-1,2-dihydro-3H-indol-3-ylidene)methyl]phenoxy}phenyl) propanoate hydrochloride (1.0 gm, 2.4 mmol) in methanol (300 ml) was added 10% Pd/C (0.5 gm). The reaction mixture was charged to hydrogenator flask and hydrogenated at 140 psi pressure for 3 hr. The progress of reaction was monitored by HPLC. After completion of reaction, the reaction mixture was filtered, solvent was evaporated under reduced pressure to yield a pale yellow solid. Yield: 0.85 gm; ¹HNMR (DMSO-d₆ 400 MHz): δ 2.9 (m, 1H), 3.0 (m, 2H), 3.2 (m, 1H), 3.6 (s, 3H), 3.7 (m, 1H), 4.2 (m, 1H), 6.7 (d, 1H), 6.8 (m, 2H), 6.9 (m, 3H),7.1 (m,4H), 7.2 (d,2H),), 7.9 (bs,2H), 10.3 (s,1H). m/z^{m+1}: 417.3.

### EXAMPLE 3

Synthesis of Methyl-2-amino-3-(4-{4-[(2-oxo-1,2-dihydro-3H-indol-3-ylidene)methyl]phenoxy}phenyl) propanoate hydrochloride.

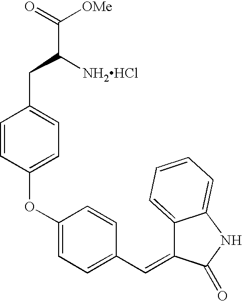

Step I

Preparation of (2S)-2-[(tert-butoxycarbonyl)amino]-3-[4-(4-formylphenoxy)phenyl]propanoic acid.

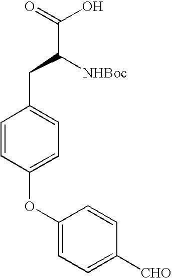

To the suspension of potassium carbonate (122.9 gm, 890 mmol) in dry DMF (150 ml), under nitrogen atmosphere charged Boc-tyr-OH (50 gm, 177 mmol) at 30° C. Reaction mixture was strirred for 15 minutes then p-Fluorobenzaldehyde solution (110.3 gm, 889 mmol) was added. The reaction mixture was stirred for 24 hr at 80° C. The TLC shows absence of BOC-Tyr-OH. The solvent was distilled under high vacuum. The thick reaction mass obtained was quenched with 0.5M NaOH solution. The reaction mixture was extracted with ethyl acetate. Aqueous layer was acidified to pH 2 using 2N HCl, extracted with ethyl acetate (2×400 ml). Combined organic layer was dried over sodium sulfate and concentrated to give sticky mass. Finally the crude product was purified by column chromatography to yield the title compound. Yield 60.9 gm; ¹H NMR (CDCl₃, 400 MHz): 1.42 (s, 9H), 2.90 (m,1H), 2.97 (m, 1H), 4.61 (m, 1H), 5.00 (m, 1H), 7.03 (m, 4H), 7.23 (m, 2H), 7.83 (m, 2H), 9.92 (s, 1H); m/z^{M+1} 386.1.

Step II

Preparation of methyl (2S)-2-[(tert-butoxycarbonyl)amino]-3-[4-(4-formylphenoxy)phenyl]propanoate

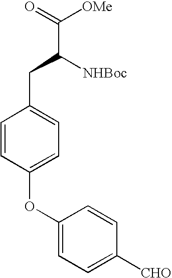

To a suspension of sodium bicarbonate (3.36 gm, 3.98 mmol) in anhydrous DMF (10 ml) 2-tert-butoxycarbonylamino-3-{4-[4-(2-oxo-1,2-dihydroindol-3lidenemethyl)-phenoxy]-phenyl}-propionic acid (1.0 gm, 2.59 mmol) and methyl iodide (0.62 ml, 9.98 mmol) was added and stirred overnight at room temperature. A solution of potassium hydroxide (0.5M, 10 ml) was added to the reaction mixture and extracted with ethyl acetate. Organic layer was washed with water, brine, dried over anhydrous magnesium sulfate and evaporated to yield the title compound. Yield :0.9 gm; ¹H NMR (DMSO-d₆, 400 MHz,): 1.42 (S, 9H) 3.2 (m,2H), 3.73 (s,3H), 4.65 (m, 1H), 5.05 (m, 1H), 7.03 (m, 4H) 7.17 (d, 2H), 7.83 (d, 2H), 9.9 (S, 1H); m/z^{M+1} 400.2.

Step III

Preparation of Methyl-2-[(tert-butoxycarbonyl)amino]-3-(4-{4-[(2-oxo-1,2-dihydro-3H-indol-3-ylidene)methyl]phenoxy}phenyl) propanoate.

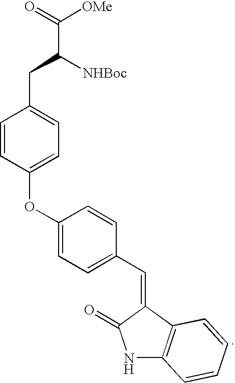

A solution of methyl (2S)-2-[(tert-butoxycarbonyl)amino]-3-[4-(4-formyl phenoxy)phenyl]propanoate (2.0 gm, 5.0 mmol), 2-oxo-indole (0.8 gm , 6.0 mmol), benzoic acid (0.091 gm, 0.75 mmol) and piperidine (0.055 gm, 0.65 mmol) in toluene (50 ml) was refluxed with stirring at 145°-155° C. with continuous removal of water using dean stark apparatus for 3 hr. The reaction mixture was allowed to attain room temperature and concentrated. The crude product thus obtained was purified by column chromatography. Yield: 1.8 gm; m/z^{M+1} 400.2.

Step IV

Preparation of Methyl-2-amino-3-(4-{4-[(2-oxo-1,2-dihydro-3H-indol-3-ylidene)methyl]phenoxy}phenyl) propanoate hydrochloride.

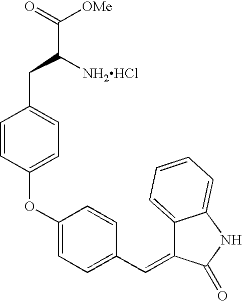

Dry HCl gas was passed slowly to the solution of Methyl-2-[(tert-butoxycarbonyl) amino]-3-(4-{4-[(2-oxo-1,2-dihydro-3H-indol-3-ylidene)methyl]phenoxy}phenyl) propanoate (0.8 gm, 1.5 mmol) in dichloromethane (100 ml) at 0 to 5° C. for 2 hrs. After completion of the reaction, the excess of hydrochloric acid gas was removed by bubbling nitrogen gas. The solid thus separated out was filtered, washed with dichloromethane and dried to furnish the titled product. Yield: 0.62 gm; ¹H NMR DMSO-d₆, 400 MHz) δppm: 3.15 (m,2H), 3.72 (s,3H), 4.33 (m,1H), 6.87 (m,2H), 7.11 (m,4H), 7.23 (m,1H), 7.32 (m,2H), 7.6 (m,2H), 7.77 (m,2H), 8.55 (bs,2H), 10.6 (s,1H); m/z^{M+1} 415.

The following compounds were prepared according to the procedure given in example 3.

| Example | | |
|---|---|---|
| No. | Structure | Analytical Data |
| 4 | 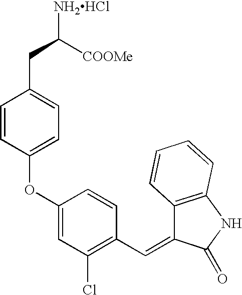 | Yield: 0.42 gm (¹HNMR, DMSO-d₆400 MHz): δ3.1 (d, 2 H), 3.7 (s, 3 H), 4.35 (m,1 H), 6.8 (m, 2 H)1, 7.0 (m, 1 H), 7.1 (m, 3 H),7.2 (m, 1 H), 7.25 (m, 1 H), 7.5 (m, 2 H), 7.53(s, 1 H), 7.8 (d, 1 H), 8.45 (bs, 2 H), 10.5 (s,1 H);m/z^{M+1}: 449.1. |
| 5 | 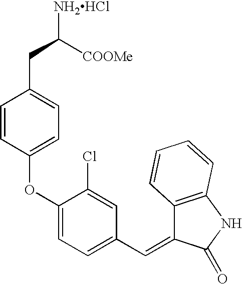 | Yield: 0.45 gm (¹HNMR, DMSO-d₆400 MHz): δ 3.1 (d, 2 H), 3.7 (s, 3 H), 4.3 (m,1 H), 6.88 (d, 2 H), 7.1 (q, 3 H), 7.2 (t, 1 H), 7.3(m, 2 H), 7.5 (d, 1 H), 7.57 (s, 1 H), 7.73 (m,1 H), 7.9 (m, 1 H), 8.5 (bs, 2 H), 10.6 (s, 1 H);m/z^{M+1}: 449.1. |
| 6 | 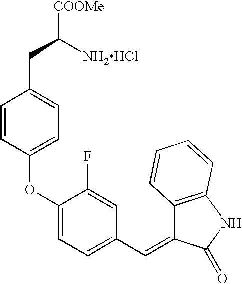 | Yield: 0.60 gm (¹HNMR, DMSO-d₆400 MHz): δ 3.1 (d, 2 H), 3.7 (s, 3 H), 4.3 (m,1 H), 6.8 (m, 2 H), 7.0 (m, 3 H), 7.2 (m, 4 H),7.5 (m, 3 H), 8.5 (bs, 2 H), 10.62 (s, 1 H);m/z^{M+1}: 433.5. |
| 7 | 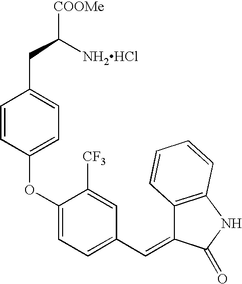 | Yield: 0.52 gm (¹HNMR, DMSO-d₆400 MHz): δ 3.1(m, 2 H), 3.7 (s, 3 H), 4.3 (m,1 H), 6.8 (m, 2 H), 7.0 (d, 1 H), 7.1 (d, 2 H), 7.2(m, 1 H), 7.3 (d, 1 H), 7.4 (m, 1 H), 7.6 (s, 1 H),8.0 (d, 1 H), 8.1 (s, 1 H), 8.5 (bs, 2 H), 10.6(s, 1 H);m/z^{M+1}: 483.3. |
| 8 | 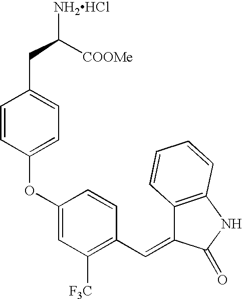 | Yield: 1.25 gm (¹HNMR, DMSO-d₆400 MHz): δ3.1 (m, 2 H), 3.7 (s, 3 H), 4.3 (t, 1 H),6.8 (d, 1 H), 6.88 (d, 1 H), 7.0 (d, 1 H), 7.2(m, 3 H), 7.3 (m, 3 H), 7.4 (s, 1 H), 7.6 (d, 1 H),7.8 (d, 1 H), 8.5 (bs, 2 H), 10.7(s, 1 H);m/z^{M+1}: 483.4 |
| 9 | 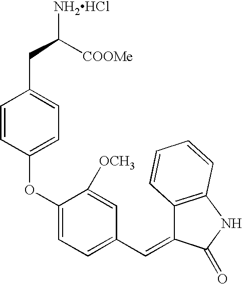 | Yield: 0.4 gm (¹HNMR, DMSO-d₆400 MHz): δ3.1 (m, 2 H), 3.7 (s, 3 H), 3.80 (s,3 H), 4.3 (m, 1 H), 6.9 (m, 4 H), 7.08 (d, 1 H), 7.2(m, 3 H), 7.53 (d, 1 H), 7.6 (s, 1 H), 7.68 (s, 1 H),7.7 (d, 1 H), 8.5 (bs, 2 H), 10.64 (s, 1 H).m/z^{M+1}: 445.2 |

### EXAMPLE 10

Synthesis of Methyl-2-amino-3-(4-{4-[(2-oxo-2,3-dihydro-1H-indol-3-yl)methyl]phenoxy}phenyl) propanoate hydrochloride.

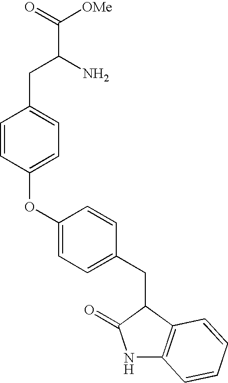

To the solution of Methyl-2-amino-3-(4-{4-[(2-oxo-1,2-dihydro-3H-indol-3-ylidene)methyl]phenoxy}phenyl)propanoate hydrochloride (0.6 gm, 1.4 mmol) in methanol (0.3 ml) was added 10% Pd/C (0.4 gm). The reaction mixture was charged to hydrogenator flask and hydrogenated at 140 psi pressure for 3 hr. The progress of reaction was monitored by HPLC. After completion of reaction the reaction mixture was filtered , solvent was evaporated under reduced pressure to yield a pale yellow solid. Yield: 0.50 gm, (¹HNMR DMSO-d₆ 400 MHz ): δ 2.9 (m, 1H), 3.0 (m, 2H), 3.2 (m, 1H), 3.6 (s, 3H), 3.7 (m, 1H), 4.2 (m, 1H), 6.7 (d, 1H), 6.8 (m, 2H), 6.9 (m, 3H),7.1 (m,4H), 7.2 (d,2H),). m/z^{m+1}: 417.0.

The following compounds were prepared according to the procedure given in example 10

| | | |
|---|---|---|
| 11 | 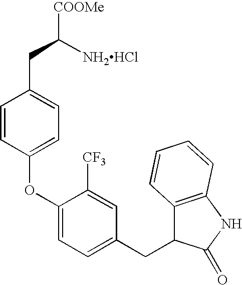 | Yield: 0.22 gm (¹HNMR, DMSO-d₆400 MHz): δ3.0 (d, 2 H), 3.1 (m, 1 H), 3.3 (m,1 H), 3.6 (s, 3 H), 3.8 (t, 1 H), 4.0 (t, 1 H), 6.7 (d, 1 H),6.8 (d, 1 H), 6.9 (m, 3 H), 7.1 (m, 2 H), 7.2 (d,1 H), 7.3 (d, 2 H), 7.4 (s, 1 H), 8.5 (bs, 2 H), 10.6(s, 1 H);m/z^{M+1}: 485.4 |
| 12 | 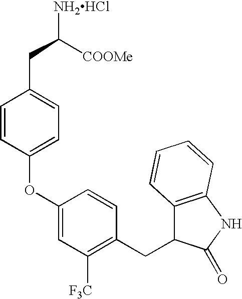 | Yield: 0.6 gm (¹HNMR, DMSO-d₆400 MHz): δ 2.9(m, 1 H), 3.1(m, 3 H), 3.7(s, 3 H),3.8(m, 1 H), 4.2(m, 1 H), 6.6(d, 1 H), 6.8(m,2 H), 7.0(d, 1 H), 7.2(m, 6 H), 7.6(d, 1 H), 8.5(bs,2 H), 10.5(s, 1 H).m/z^{M+1}: 485.3 |
| 13 | 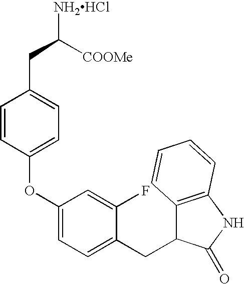 | Yield: 0.35 gm (¹HNMR DMSO-d₆400 MHz): δ 2.5(m, 1 H), 2.8(m, 2 H), 3.1(m, 1 H),3.2(m, 1 H), 3.7(s, 3 H), 4.2(t, 1 H), 6.6(m,1 H), 6.7(m, 4 H), 6.9(d, 1 H), 7.0(d, 1 H), 7.1(m,1 H), 7.2(m, 3 H), 8.3 (bs, 2 H), 10.4(s, 1 H).m/z^{M+1}: 435.2 |
| 14 | 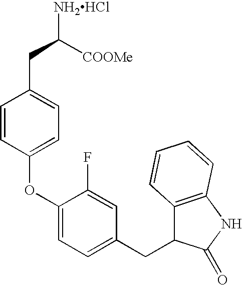 | Yield: 0.46 gm (¹HNMR DMSO-d₆400 MHz): δ 3.0(m, 3 H), 3.1(s, 1 H), 3.3(m, 1 H),3.7(s, 3 H), 4.2(t, 1 H), 6.8(m, 3 H), 6.9(m,3 H), 7.0(d, 1 H), 7.1(m, 4 H), 8.5(bs, 2 H), 10.4(s, 1 H).m/z^{M+1}: 435.3 |
| 15 | 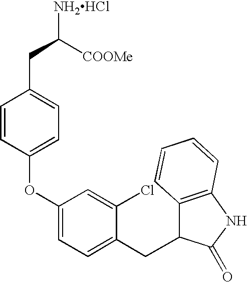 | Yield: 0.38 gm (¹HNMR DMSO-d₆400 MHz): δ 2.9(m, 1 H), 3.1(m, 2 H), 3.3(m, 1 H),3.7(s, 3 H), 3.78(m, 1 H), 4.3(m, 1 H), 6.8(m,2 H), 6.9(m, 2 H), 7.0(m, 2 H), 7.1(m, 2 H), 7.2(d, 1 H), 7.29(d, 1 H), 7.3(d, 1 H), 8.4(bs, 2 H),10.4(s, 1 H).m/z^{M+1}: 451.2 |
| 16 | 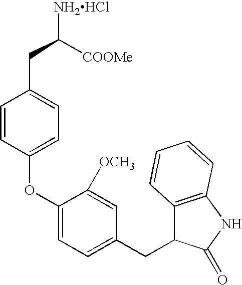 | Yield: 0.19 gm (¹HNMR DMSO-d₆400 MHz): δ 2.96(m, 1 H), 3.02(m, 2 H), 3.16(d,1 H), 3.35(s, 3 H), 3.59(s, 3 H), 3.68(m, 1 H), 4.17(t, 1 H), 6.72(m, 4 H), 6.83(m, 1 H), 6.87(m, 1 H),6.93(d, 2 H), 7.12(m, 3 H), 8.11(bs, 2 H),10.34(s, 1 H).m/z^{M+1}: 447.1 |
| 17 | 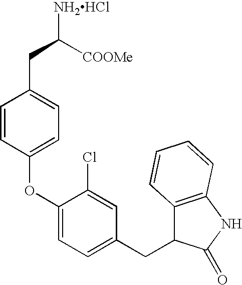 | Yield: 0.28 gm (¹HNMR DMSO-d₆400 MHz): δ 3.09(m, 2 H), 3.16(d, 1 H), 3.26(m,1 H), 3.69(s, 3 H), 3.84(m, 1 H), 4.27(t, 1 H), 6.77(m,1 H), 6.82(m, 2 H), 6.9(m, 3 H), 7.13(d, 3 H), 7.22(d,2 H), 8.59(bs, 2 H), 10.4(s, 1 H).m/z^{m +1}: 451.2 |

### EXAMPLE 18

Synthesis of 2-amino-3-{4-[4-(2-oxo-1,2-dihydroindol-3-ylidenemethyl)-phenoxy]-phenyl}-propionic acid hydochloric acid.

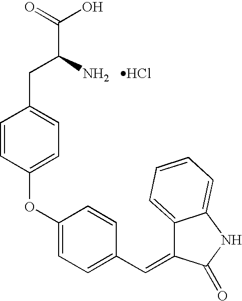

Step I

Preparation of 2-tert-butoxycarbonylamino-3-{4-[4-(2-oxo-1,2-dihydroindol-3-ylidenemethyl)-phenoxy]-phenyl}-propionic acid.

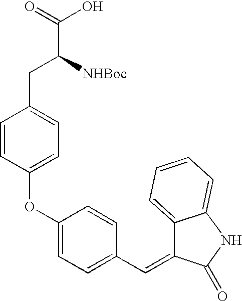

Suspended (2S)-2-[(tert-butoxycarbonyl)amino]-3-[4-(4-formylphenoxy)phenyl]propanoic acid (3.6 gm, 9.35 mmol) in toluene (30.0 ml) and to this was added benzoic acid (0.17 gm, 1.4 mmol), piperidine (1.06 ml, 10.75 mmol) and oxindole (1.49 gm, 11.2 mmol) and heated at 130° C. with continuous removal of water. Toluene was evaporated under reduced pressure. The residue obtained was taken up in ethyl acetate (100 ml) acidified with 2M HCl. Organic layer was washed with water, brine (1×50 ml) and dried over anhydrous magnesium sulfate. The crude product obtained was purified by silica gel chromatography using hexane-ethyl acetate (3:7) containing 1% acetic acid to yield, 2-tert-butoxycarbonylamino-3-{4-[4-(2-oxo-1,2-dihydroindol-3-ylidenemethyl)-phenoxy]-phenyl}-propionic acid as yellow solid, 3.6 gm, ¹H NMR (DMSO-d₆): 7.75 (d, J=8.8 Hz, 2H), 7.59 (s, 1H), 7.32 (overlapped d, 4H), 7.06 (m, 4H), 6.83 (m, 2H), 4.12 (m, 1H), 3.05 (dd, J=14.0 and 4.4 Hz, 1H), 2.83 (dd, J=14.0 and 10.8 Hz, 1H), 1.34 (s, 9H).

Step II

Preparation of 2-amino-3-{4-[4-(2-oxo-1,2-dihydroindol-3-ylidenemethyl)-phenoxy]-phenyl}-propionic acid hydochloric acid.

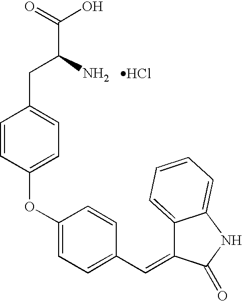

Compound 2-tert-butoxycarbonylamino-3-{4-[4-(2-oxo-1,2-dihydroindol-3-ylidenemethyl)-phenoxy]-phenyl}-propionic acid (1.5 gm) was dissolved in dichloromethane (20 ml), cooled in an ice bath and passed HCl gas for 30 min. Ice bath was removed and stirred the suspension at room temperature for another 20 min. Excess HCl was removed evacuating the suspension and purging with argon. Dichloromethane was evaporated under reduced pressure and solid obtained was washed with triturated with ethyl acetate to yield 2-amino-3-{4-[4-(2-oxo-1,2-dihydroindol-3-ylidenemethyl)-phenoxy]-phenyl}-propionic acid hydochloric acid salt (0.9 gm) as yellow solid. ¹H NMR (DMSO-d₆): 10.61 (br, 1H), 7.76 (d, J=8.4 Hz, 2H), 7.60 (s, 1H), 7.35 (d, J=8.8 Hz, 2H), 7.24 (m, 1H), 7.12 (m, 4H), 7.04 (m, 1H), 6.88 (d, J=8.0 Hz, 2H), 4.17 (t, J=6.4 Hz, 1H), 3.17 (dd, J=14.0 and 6.0 Hz, 1H), 3.08 (dd, J=14.0 and 7.2 Hz, 1H).

### EXAMPLE 19

Synthesis of 2-amino-3-{4-[4-(2-oxobenzothiazol-3-ylmethyl)-phenoxy]-phenyl}-propionic acid methyl ester hydrochloric acid.

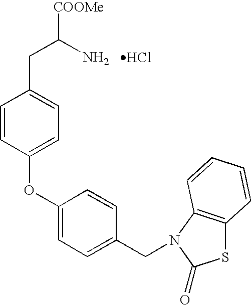

Step I

Preparation of 2-tert-butoxycarbonylamino-3-{4-[4-(2-oxobenzothiazol-3-ylmethyl)-phenoxy]-phenyl}-propionic acid methyl ester.

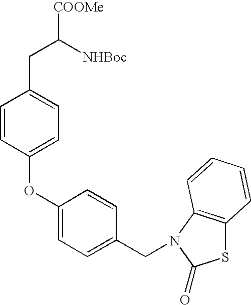

To a suspension of sodium hydride (9.5 gm, 0.23 mmol) DMF (5 ml) in methyl-2-N-Boc-amino-3-[4-(4-{[(methylsulfonyl)oxy]methyl}phenoxy)phenyl]propanoate (0.11 g, 0.23 mmol) and 2-oxo-benzothiazole (0.035 gm, 0.23 mmol) were added and heated at 60° C. for 5 h. Mixture was poured in saturated ammonium chloride solution and extracted with dichloromethane . Organic layer was washed with water, brine, dried over anhydrous magnesium sulfate and evaporated. Crude product was purified by silica gel chromatography to yield 2-tert-butoxycarbonylamino-3-{4-[4-(2-oxobenzothiazol-3-ylmethyl)-phenoxy]-phenyl}-propionic acid methyl ester (0.065 gm).

Step II

Preparation of 2-amino-3-{4-[4-(2-oxobenzothiazol-3-ylmethyl)-phenoxy]-phenyl}-propionic acid methyl ester hydrochloric acid.

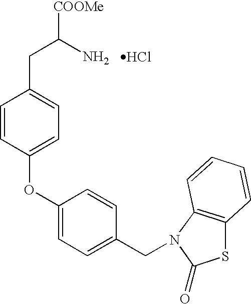

2-tert-butoxycarbonylamino-3-{4-[4-(2-oxobenzothiazol-3-ylmethyl)-phenoxy]-phenyl}-propionic acid methyl ester (0.059 gm) was dissolved in CH₂Cl₂ (10 ml) and cooled to 0-5° C. Hydrogen chloride gas was bubbled through this solution for 20 min. The bubbling was discontinued and the reaction mixture was stirred at room temperature for 1 h. The excess HCl was degassed and the CH₂Cl₂ was removed. The residual solid was triturated with anhydrous diethyl ether, decanted, and dried to yield the desired compound 2-amino-3-{4-[4-(2-oxobenzothiazol-3-ylmethyl)-phenoxy]-phenyl}-propionic acid methyl ester hydrochloric acid as a solid (0.02 gm). ¹H NMR (400 MHz, DMSO-d₆): 8.37 (br, 2H), 7.69 (d, J=8.0 Hz, 1H), 7.33-7.37 (overlapped d, 4H), 7.18-7.23 (m, 3H), 6.94-6.98 (overlapped d, 4H), 5.17 (s, 2H), 4.28 (t, J=6.4 Hz, 1H), 3.60 (s, 3H), 3.07 (d, J=6.4 Hz, 2H).

### EXAMPLE 20

Synthesis of 2-amino-N,N-dimethyl-3-{4-[4-(2-oxo-1,2-dihydroindol-3-ylidenemethyl)-phenoxy]-phenyl}-propionamide hydrochloric acid.

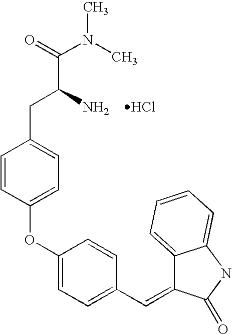

Step I

Preparation of (1-dimethylcarbamoyl-2-{4-[4-(2-oxo-1,2-dihydroindol-3-ylidenemethyl)-phenoxy]-phenyl}-ethyl)-carbamic acid tert-butyl ester.

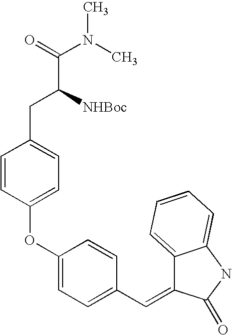

Compound 2-tert-butoxycarbonylamino-3-{4-[4-(2-oxo-1,2-dihydroindol-3-ylidenemethyl)-phenoxy]-phenyl}-propionic acid (1.0 gm, 1.99 mmol) was dissolved in CH₂Cl₂ (25 ml)and stirred at room temperature under an atmosphere of argon. Triethylamine (0.67 ml, 4.8 mmol) and benzotriazol-1-yloxy-tris(dimethylamino)phosphonium hexafluorophosphate (BOP reagent, 1.96 gm, 4.4 mmol) were added and the reaction mixture was stirred for 15 min. Dimethylamine (2.0 M solution in THF, 10.0 ml, 20.0 mmol) was added and the resulting solution was stirred overnight at room temperature. The solvent was removed under reduced pressure and the resulting residue was taken up in EtOAc (50 ml). The organic layer was extracted with 1.0 N NaOH (1×10 ml), water (2×30 ml) and brine (1×30 ml). Drying and concentration of the organic layer gave the desired (1-dimethylcarbamoyl-2-{4-[4-(2-oxo-1,2-dihydroindol-3-ylidenemethyl)-phenoxy]-phenyl}-ethyl)-carbamic acid tert-butyl ester. (1.0 gm, 95.2%). ¹H NMR (400 MHz, DMSO-d₆): 7.59-7.76 (m, 3H), 7.30-7.35 (nm, 2H), 6.66-7.17 (m, 8H), 4.60 (m, 1H), 2.94 (s, 3H), 2.75-2.90 (m, 5H), 1.32 (s, 9H).

Step II

Preparation of 2-amino-N,N-dimethyl-3-{4-[4-(2-oxo-1,2-dihydroindol-3-ylidenemethyl)-phenoxy]-phenyl}-propionamide hydrochloric acid.

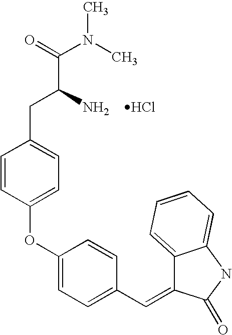

The compound (1-dimethylcarbamoyl-2-{4-[4-(2-oxo-1,2-dihydroindol-3-ylidenemethyl)-phenoxy]-phenyl}-ethyl)-carbamic acid tert-butyl ester (1.0 gm) was dissolved in CH₂Cl₂ (20 ml) and cooled to 0-5° C. Hydrogen chloride gas was bubbled through this solution for 20 min. The bubbling was discontinued and the reaction mixture was stirred at room temperature for 1 h. The excess HCl was degassed and the CH₂Cl₂ was removed. The residual solid was triturated with EtOAc (2×20 ml), decanted, and dried to yield the desired compound 2-amino-N,N-dimethyl-3-{4-[4-(2-oxo-1,2-dihydroindol-3-ylidenemethyl)-phenoxy]-phenyl}-propionamide hydrochloric acid salt as a yellow amorphous solid (0.8 gm, 90.9%). ¹H NMR (DMSO-d₆): 7.60-7.83 (m, 3H), 7.30-7.33 (m, 2H), 6.87-7.24 (m, 8H), 4.62 (m, 1H), 3.01 (m, 2H), 2.85 (s, 3H), 2.79 (s, 3H).

### EXAMPLE 21

Synthesis of 2-amino-3-{4-[4-(2-oxo-1,2-dihydroindol-3-ylidenemethyl)-phenoxy]-phenyl}-propionamide hydrochloric acid.

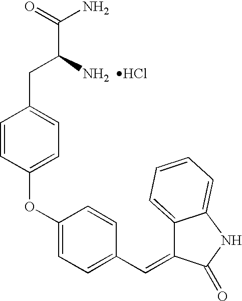

Step I

Preparation of (1-carbamoyl-2-{4-[4-(2-oxo-1,2-dihydroindol-3-ylidenemethyl)-phenoxy]-phenyl}-ethyl)-carbamic acid tert-butyl ester.

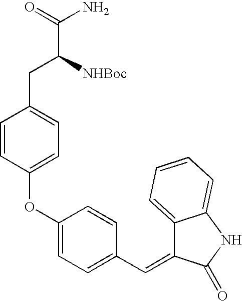

The compound 2-tert-butoxycarbonylamino-3-{4-[4-(2-oxo-1,2-dihydroindol-3-ylidenemethyl)-phenoxy]-phenyl}-propionic acid (1.0 gm, 1.99 mmol) was dissolved in CH₂Cl₂ (25 ml) and stirred at room temperature under an atmosphere of argon. Triethylamine (0.67 ml, 4.8 mmol) and BOP reagent (1.6 g, 4.4 mmol) were added and the reaction mixture was stirred for 15 min. Ammonia was bubbled to this solution for 30 min and stirred overnight at room temperature. The solvent was removed under reduced pressure and the resulting residue was taken up in EtOAc (100 ml). The organic layer was extracted with 0.5 N NaOH (1×25 ml), water (2×30 ml) and brine (1×30 ml). Drying and concentration of the organic layer gave the desired compound (1-carbamoyl-2-{4-[4-(2-oxo-1,2-dihydroindol-3-ylidenemethyl)-phenoxy]-phenyl}-ethyl)-carbamic acid tert-butyl ester (1.0 gm, quantitative). ¹H NMR (400 MHz, DMSO-d₆): 7.74 (d, J=9.2 Hz, 2H), 7.59 (s, 1H), 7.40 (br, 1H), 7.34 (m, 2H), 7.07 (m, 4H), 6.88 (m, 2H), 4.10 (m, 1H), 2.98 (m, 1H), 2.75 (m, 1H), 1.31 (s, 9H).

Step II

Preparation of 2-amino-3-{4-[4-(2-oxo-1,2-dihydroindol-3-ylidenemethyl)-phenoxy]-phenyl}-propionamide hydrochloric acid salt.

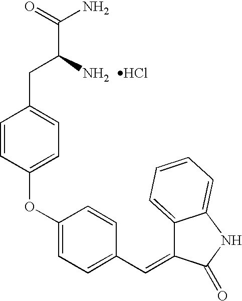

The compound (1-carbamoyl-2-{4-[4-(2-oxo-1,2-dihydroindol-3-ylidenemethyl)-phenoxy]-phenyl}-ethyl)-carbamic acid tert-butyl ester (1.0 gm) was dissolved in CH₂Cl₂ (20 ml) and cooled to 0-5° C. Hydrogen chloride gas was bubbled through this solution for 20 min. The bubbling was discontinued and the reaction mixture was stirred at room temperature for 1 h. The excess HCl was degassed and the CH₂Cl₂ was removed. The residual solid was triturated with EtOAc (2×20 ml), decanted, and dried to yield the desired compound 2-amino-3-{4-[4-(2-oxo-1,2-dihydroindol-3-ylidenemethyl)-phenoxy]-phenyl}-propionamide hydrochloric acid salt as a yellow amorphous solid (0.8 gm, 92.0%). ¹H NMR (DMSO-d₆): 7.77 (m, 2H), 7.61 (m, 3H), 7.36 (m, 2H) 7.11 (m, 4H), 6.87 (m, 1H), 6.54 (s, 1H), 3.95 (m, 1H), 3.12 (m, 1H). 2.97 (m, 1H).

The pharmaceutically acceptable salts are prepared by reacting the compound of formula (I) with 1 to 4 equivalents of a base such as sodium hydroxide, sodium methoxide, sodium hydride, potassium t-butoxide, calcium hydroxide, magnesium hydroxide and the like, in solvents like ether, THF, methanol, t-butanol, dioxane, isopropanol, ethanol etc. Mixtures of solvents may be used. Organic bases like lysine, arginine, diethanolamine, choline, guanidine and their derivatives etc. may also be used. Alternatively, acid addition salts are prepared by treatment with acids such as hydrochloric acid, hydrobromic acid, nitric acid, sulfuric acid, phosphoric acid, p-toluenesulfonic acid, methanesulfonic acid, acetic acid, citric acid, maleic acid, salicylic acid, hydroxynaphthoic acid, ascorbic acid, palmitic acid, succinic acid, benzoic acid, benzene sulfonic acid, tartaric acid and the like in solvents like ethyl acetate, ether, alcohols, acetone, THF, dioxane etc. Mixture of solvents may also be used.

The present invention also provides a pharmaceutical composition, containing one or more of the compounds of the general formula (I) as defined above, their pharmaceutically acceptable salts in combination with the usual pharmaceutically employed carriers, diluents and the like.

The pharmaceutical composition may be in the forms normally employed, such as tablets, capsules, powders, syrups, solutions, suspensions and the like, may contain flavourants, sweeteners etc. in suitable solid or liquid carriers or diluents, or in suitable sterile media to form injectable solutions or suspensions. Such compositions typically contain from 1 to 25%, preferably 1 to 15% by weight of active compound, the remainder of the composition being pharmaceutically acceptable carriers, diluents, excipients or solvents.

Suitable pharmaceutically acceptable carriers include solid fillers or diluents and sterile aqueous or organic solutions. The active compound will be present in such pharmaceutical compositions in the amounts sufficient to provide the desired dosage in the range as described above. Thus, for oral administration, the compounds can be combined with a suitable solid or liquid carrier or diluent to form capsules, tablets, powders, syrups, solutions, suspensions and the like. The pharmaceutical compositions, may, if desired, contain additional components such as flavourants, sweeteners, excipients and the like. For parenteral administration, the compounds can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable acid addition salts or alkali or alkaline earth metal salts of the compounds. The injectable solutions prepared in this manner can then be, administered intravenously, intraperitoneally, subcutaneously, or intramuscularly, with intramuscular administration being preferred in humans.

The pharmaceutical composition of the present invention are effective in lowering blood glucose, serum insulin and triglyceride levels in animal models of types II diabetes. The pharmaceutical compositions of the present invention are also effective in the treatment of obesity, inflammation, and autoimmune diseases. Furthermore, pharmaceutical composition of the present invention are useful for the treatment of disorders associated with insulin resistance, such as polycystic ovary syndrome, as well as hyperlipidemia, coronary artery disease and peripheral vascular disease, and for the treatment of inflammation and immunological diseases, particularly those mediated by cytokines such as TNF-α, IL-1, IL-6 and cyclooxygenase such as COX-2.

Protocols for Biological Testing

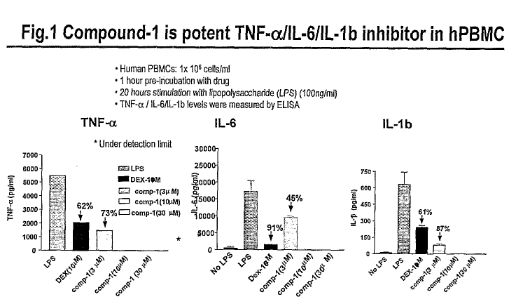

FIG. 1. TNF-α, IL-6 and IL-1β inhibition in human peripheral blood monocytic cells (hPBMC).

Compound 1 in example 1 inhibits major pro-inflammatory cytokines in human peripheral blood mononuclear cells isolated from volunteers. Human PBMC cells were cultured and incubated with compound 1 in example 1 at different concentrations. Cells (1×10⁶/mL) were challenged with lipopolysaccharides (LPS) at a concentration of (100 ng/mL) for 20 hours. Cell supernatant was analyzed for the presence of TNF-α, IL-1β and IL-6 cytokines by antibody directed enzyme-linked immunoassay. As shown in FIG. 1, the example compound can inhibit the production of three major pro-inflammatory cytokines in a dose dependent manner. No significant change in cell viability was observed with incubation of cells in the presence of highest concentration of the compound. These strongly indicate that compound 1 in example 1 is highly effective in reducing the production of pro-inflammatory cytokines.

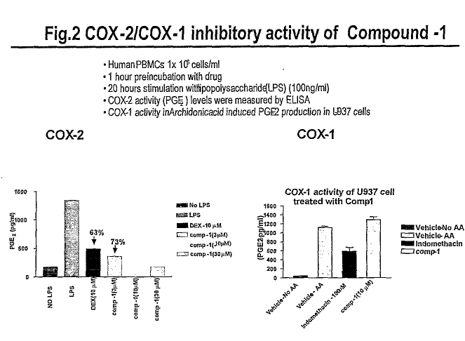

FIG. 2. Compound 1 in Example 1 Selectively Inhibits COX-2 Than COX-1 Enzyme.

Compound 1 in example 1 inhibits major pro-inflammatory cytokines in human monocyte cells as described in the FIG. 1. The inflammatory stimulus, LPS, also induces cycloxygenase-2 (COX-2) enzyme in this system and as a result prostaglandin E 2 (PGE-2) is produced. Human PBMC cells were cultured and incubated with compound in example 1 at different concentrations. Cells (1×10⁶/mL) were challenged with lipopolysaccharides (LPS) at a concentration of (100 ng/mL) for 20 hours. Inhibition of COX-2 was determined by the measurement of PGE-2 levels in the supernatant by ELISA (Cayman Chemicals). For the determination of PGE-2 levels, supernatants were incubated in the ELISA plate and detected by calorimetric reaction. Compound 1 in example 1 dose dependently inhibited LPS induced COX-2 activity in this cell types. The endogenous form, COX-1 is only activated when there is a physiological stimulus and goes through the same pathway for the production of PGE-2 levels. Human monocytic U-937 (2.5×10⁵/mL) cells were charged with arachidonic acid to activate the COX-1 pathway. It produced PGE-2 following these kind of physiological induction. To assay the COX-1 activity, cells were preincubated with the compound for 15 min (indomethacin was kept as positive control) and then they were challenged with 10 μM arachidonic acid for next 20 min. Supernatants were harvested and measured for PGE-2 as marker of COX-1 activity. Although 10 μM of compound 1 in example 1 completely inhibited LPS induced PGE-2 production, it has no effect on arachidonic acid induced PGE-2 levels depicting its selectivity on COX-2 over COX-1 activity.

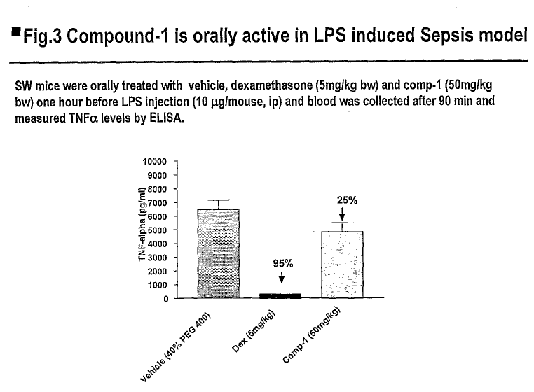

FIG. 3. Compound 1 Inhibits LPS Induced TNF-α Production In Vivo.

Swiss Webster (SW) mice were orally treated with vehicle, dexamethasone (5 mg/kg bw) and compound 1 in example 1 (50 mg/kg bw) one hour before LPS injection (10 μg/mouse, ip) and blood was collected after 90 min and measured TNF-α levels by ELISA as described in FIG. 1. Compound 1 in example 1 reduced 25% of TNF levels compared to control group of animals, whereas dexamethasone showed strong inhibition (95%) at 5 mg/kg body weight dose.

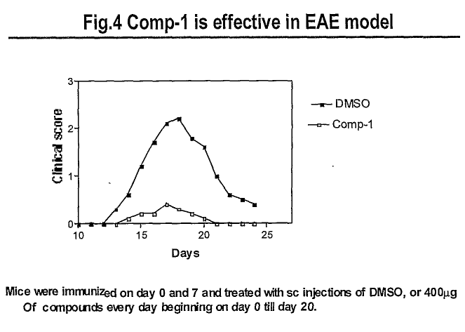

FIG. 4. Compound 1 in Example 1 Reduced the Severity of Experimental Allergic Encephalomyeletis (EAE).

Multiple Sclerosis (MS) is an autoimmune disease and is regulated by cytokine levels. In order to test the effect of compound 1 in example 1 in MS model, experimental allergic encepahalomyalitis (EAE) was induced in SJL/J mice. EAE is an autoimmune inflammatory disease of the central nervous system (CNS). The disease shows many similarities with the human MS, and hence is used as a model to test the potential efficacy of new drugs that may have applicability in MS. EAE was induced by injecting spinal chord homogenate and the animals were treated with example compounds. The severity of EAE was established by clinical scores of paralysis. As shown in FIG. 4, the compound 1 in example 1 treated group showed complete prevention of EAE. These results indicate utility of the example compounds for the treatment of MS and other neurological disorders.

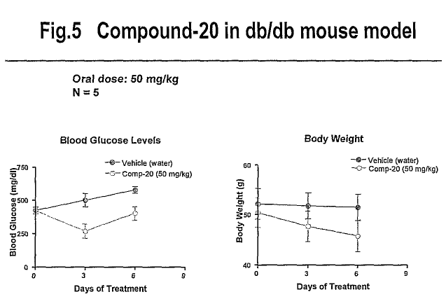

FIG. 5. Compound 20 in Example 20 Reduces Blood Glucose and Bodyweight Gain in db/db Obese Diabetic Animal Model.

Seven weeks old male db/db (spontaneous model) diabetic mice were orally treated with compound 20 in example 20 at a dose of 50 mg/kg body weight in water and blood glucose was monitored by Accuchek glucometer. This compound has also shown the lowering of body weight compared to vehicle. The results are shown in FIG. 5.

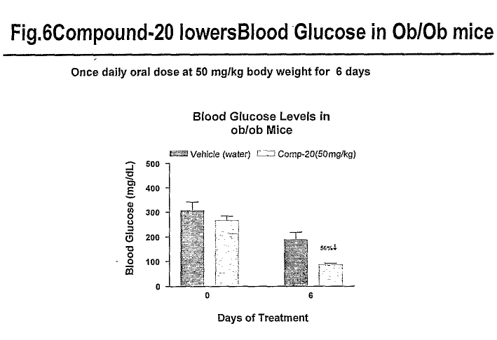

FIG. 6. Lowering of Blood Glucose in ob/ob Mice by Compound 20 in Example 20.

Seven weeks old C57BL/6J male ob/ob (obese, insulin resistant spontaneous model of type-II diabetes) diabetic mice were orally treated with compound 20 in example 20, at a dose of 50 mg/kg body weight dissolved in water and blood glucose (FIG. 6) was monitored by Accuchek glucometer. Compound 20 in example 20 show strong glucose lowering activity in this animal model of Type-II diabetes within six days of treatment.

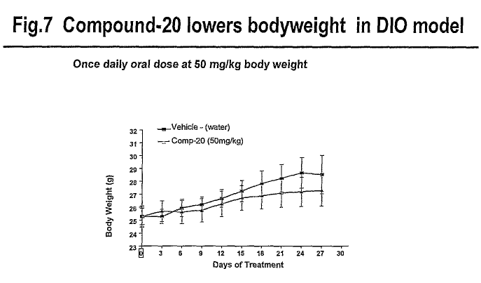

FIG. 7. Effect of Compound 20 in Example 20 on Diet Induced Obesity Mouse Models.

C57BL/6J male mouse were fed with 60% Kcal high fat diet (D12492, Research Diet) ad libitum 15 days prior to the beginning of the treatment. Diet induced obese mice were treated with a dose of 50 mg/kg body weight compound 20 in example 20 and their bodyweight was monitored on every third day. The treated animals showed less body weight gain compared to vehicle treated animals.

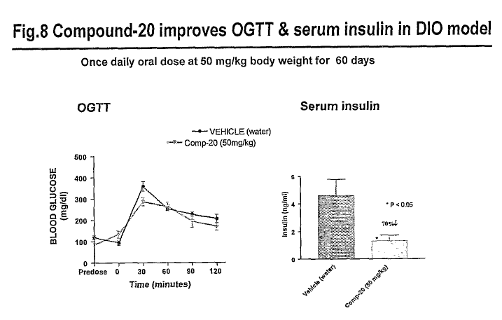

FIG. 8. Oral Glucose Tolerance Test (OGTT) in Diet Induced Obesity Model for Compound 20 in Example 20.

After 60 days of high fat feeding, these animals get hyperinsulinemic and shows impaired glucose tolerance. To see the effect of compound 20 in example 20, on day 60 an OGTT study was carried to observe the improvement of glucose tolerance upon the treatment of compound 20 in example 20. When insulin levels were measured control animals showed higher levels than treated groups. A decrease of 70% of serum insulin level was observed after compound 20 in example 20 treatment as shown in FIG. 8b.

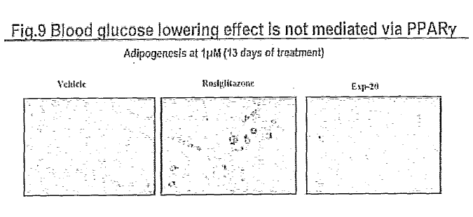

FIG. 9. Compound 20 in Example 20 is Not Adipogenic.

All known PPARγ agonists induce differentiation in fibroblast cells. The adipogenic potential of these compounds are correlated with their affinity to this receptor. To check quickly the affinity of compound 20 in example 20 to this receptors, 3T3-L1 fibroblasts were treated with either DMSO control or rosiglitazone as positive control or these two compounds for several days at different concentrations. On day 13^{th}, the differentiated adipocytes were stained with Oil-red-O (Sigma) and washed thoroughly to remove unbound stain and visualized under Olympus microscope. PPARγ agonist rosiglitazone strongly induced adipogenesis in this cell system whereas compound 20 in example 20 remained unchanged, this is the indirect proof that compound 20 in example 20 has no affinity to PPARγ receptor. The results are shown in FIG. 9.

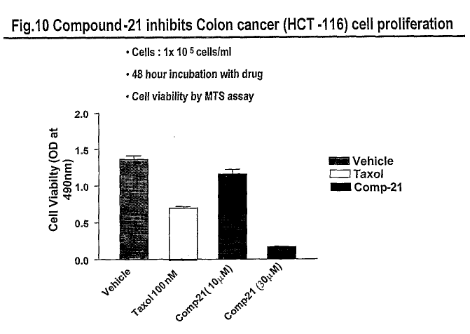

FIG. 10. Compound 21 in Example 21 Inhibits Colon Cancer Cell Growth.

HCT-116 is a human colon cancer cell line and they were grown in 96 well plates with seeding concentration of 10% cells/mL. Compound 21 in example 1 at 30 μM and Taxol (100 nM as a positive control were incubated in these cells for 48 hrs and at the end viability was checked by MTS staining (Promega). Viabile cells showed strong color development at 540 nM where was dead cells do not show any such activity. The compound 21 completely inhibited colon cancer cell growth.

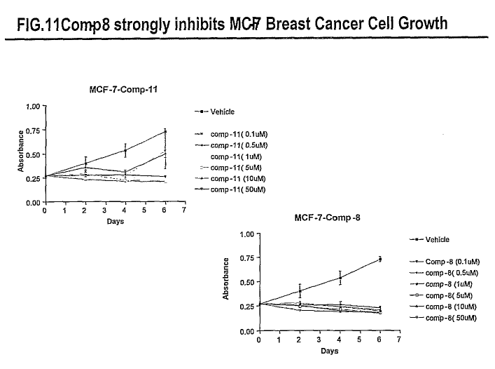

FIG. 11. Compound 11 in Example 11 and 8 in Example 8 Inhibits Breast Cancer Cell Growth.

MCF-7 is a breast cancer cells and they were grown in 96 well plates at 1000 cells/well. The cells were pretreated with either compound 11 in example 11 and 8 in example 8 or DMSO for six consecutive days. Every 48 hrs they were stained with MTS dye and viability was checked accordingly. The compound 8 in example 8 is very strong in inhibiting the breast cancer cell growth compared to 11 in example 11 as shown in FIG. 11. The doses from 1-50 μM worked equally well in case of 8 in example 8.

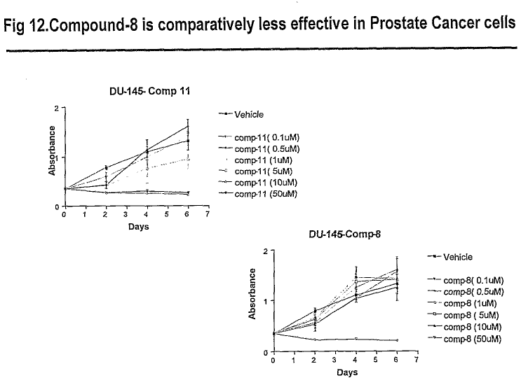

FIG. 12. Compound 11 in Example 11 and 8 in Example 8 Inhibits Prostate Cancer Cell Growth.

DU-145 is a prostate cancer cell and they were grown in 96 well plates at 1000 cells/well. The cells were pretreated with either compound 11 in example 11 and 8 in example 8 or DMSO for six consecutive days. Every 48 hrs they were stained with MTS dye and viability was checked accordingly. The compound 8 in example 8 selectively kills breast cancer cells in contrast to compound 11 in example 1. The potency of compound 11 in example 11 is very similar in both breast and prostate cancer cells.

We claim:

1. Novel tyrosine derivatives of the formula (I) 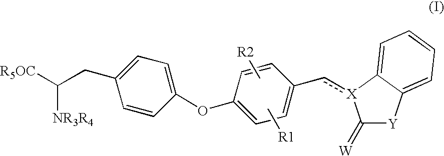
their pharmaceutically acceptable salts and their pharmaceutically acceptable compositions containing them. Wherein;
- - - represents an optional bond;
W represents O or S;
X represents C, CH or N;
Y represents NR₆, S or O;
R₁ and R₂, may be same or different and independently represent hydrogen, halogen, hydroxy, nitro, cyano, amino, alkyl, alkoxy haloalkyl;
R₃ and R₄ may be same or different and independently represent H, alkyl, COR₇;
R₅ represents OR₈, NR₉R₁₀;
R₆ represents hydrogen, substituted or unsubstituted alkyl, alkenyl, —CH₂COOR, or alyl or counter ion, wherein R represents H or alkyl group;
R₇ represents H, substituted or unsubstituted groups selected from alkyl, haloakyl alkenyl, aryl, alkenyloxy, aryloxy, alkoxy or arylalkoxy;
R₈ represents hydrogen, substituted or unsubstituted groups selected from alkyl, alkenyl, aryl, aralkyl, heteroaryl, or a counter ion;
R₉ and R₁₀ may be same or different and independently represent H, substituted or unsubstituted groups selected from alkyl, alkenyl, aryl.

2. Novel tyrosine derivatives as claimed in claim 1, wherein the - - - represent single bond or no bond.

3. Novel tyrosine derivatives as claimed in claim 1, are selected from a group comprising of:
a) Methyl-2-amino-3-(4-{4-[(2-oxo-1,2-dihydro-3H-indol-3-ylidene)methyl]phenoxy}phenyl) propanoate hydrochloride;
b) Methyl-2-amino-3-(4-{4-[(2-oxo-2,3-dihydro-1H-indol-3-yl)methyl]phenoxy}phenyl) propanoate hydrochloride;
c) Methyl-2-amino-3-(4-{4-[(2-oxo-1,2-dihydro-3H-indol-3-ylidene)methyl]phenoxy}phenyl) propanoate hydrochloride;
d) Methyl-2-amino-3-(4-{3-chloro-4-[(2-oxo-1,2-dihydro-3H-indol-3-ylidene)methyl]phenoxy}phenyl) propanoate hydrochloride;
e) Methyl-2-amino-3-(4-{2-chloro-4-[(2-oxo-1,2-dihydro-3H-indol-3-ylidene) methyl]phenoxy}phenyl)propanoate hydrochloride;
f) Methyl-2-amino-3-(4-{2-fluoro-4-[(2-oxo-1,2-dihydro-3H-indol-3-ylidene) methyl]phenoxy}phenyl) propanoate hydrochloride;
g) Methyl-2-amino-3-(4-{2-trifluoromethyl-4-[(2-oxo-1,2-dihydro-3H-indol-3-ylidene) methyl]phenoxy}phenyl) propanoate hydrochloride;
h) Methyl-2-amino-3-(4-{3-trifluoromethyl-4-[(2-oxo-1,2-dihydro-3H-indol-3-ylidene)methyl]phenoxy}phenyl) propanoate hydrochloride;
i) Methyl-2-amino-3-(4-{2-methoxy-4-[(2-oxo-1,2-dihydro-3H-indol-3-ylidene)methyl]phenoxy}phenyl) propanoate hydrochloride;
j) Methyl-2-amino-3-(4-{4-[(2-oxo-2,3-dihydro-1H-indol-3-yl)methyl]phenoxy}phenyl) propanoate hydrochloride;
k) Methyl-2-amino-3-(4-{2-trifluoromethyl-4-[(2-oxo-2,3-dihydro-1H-indol-3-yl)methyl]phenoxy}phenyl) propanoate hydrochloride;
l) Methyl-2-amino-3-(4-{3-trifluoromethyl-4-[(2-oxo-2,3-dihydro-1H-indol-3-yl)methyl]phenoxy}phenyl) propanoate hydrochloride;
m) Methyl-2-amino-3-(4-{3-fluoro-4-[(2-oxo-2,3-dihydro-1H-indol-3-yl)methyl]phenoxy}phenyl) propanoate hydrochloride;
n) Methyl-2-amino-3-(4-{2-fluoro-4-[(2-oxo-2,3-dihydro-1H-indol-3-yl)methyl]phenoxy}phenyl) propanoate hydrochloride;
o) Methyl-2-amino-3-(4-{3-chloro-4-[(2-oxo-2,3-dihydro-1H-indol-3-yl)methyl]phenoxy}phenyl) propanoate hydrochloride;
p) Methyl-2-amino-3-(4-{2-methoxy-4-[(2-oxo-2,3-dihydro-1H-indol-3-yl)methyl]phenoxy}phenyl) propanoate hydrochloride;
q) Methyl-2-amino-3-(4-{2-chloro-4-[(2-oxo-2,3-dihydro-1H-indol-3-yl)methyl]phenoxy}phenyl) propanoate hydrochloride;
r) 2-amino-3-{4-[4-(2-oxo-1,2-dihydroindol-3-ylidenemethyl)-phenoxy]-phenyl}-propionic acid hydochloride;
s) 2-amino-3-{4-[4-(2-oxobenzothiazol-3-ylmethyl)-phenoxy]-phenyl}-propionic acid methyl ester hydrochloride;
t) 2-amino-N,N-dimethyl-3-{4-[4-(2-oxo-1,2-dihydroindol-3-ylidenemethyl)-phenoxy]-phenyl}-propionamide hydrochloride;
u) 2-amino-3-{4-[4-(2-oxo-1,2-dihydroindol-3-ylidenemethyl)-phenoxy]-phenyl}-propionamide hydrochloride.

4. A pharmaceutical composition, which comprises a pharmaceutically effective amount of a novel tyrosine derivatives of formula (I) 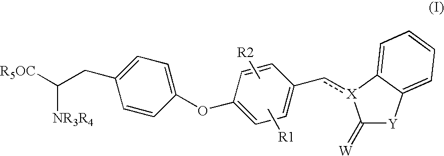
as defined in claim 1 and a pharmaceutically acceptable carrier, diluent, excipient or solvents.

5. A pharmaceutical composition as claimed in claim 3, in the form of a tablet, capsule, powder, syrup, solution, aerosol or suspension.

6. A method for reducing blood glucose, free fatty acids, cholesterol, triglycerides levels in plasma comprising administration an effective amount of a compound of formula (I) as defined in claim 1 to patient need thereof.

7. A method for treating obesity, autoimmune, inflammation, immunological, cancer disease comprising administration an effective amount of a compound of formula (I) as defined in claim 1 to patient need thereof.

8. A method for treating a disorder associated with insulin resistance comprising administrating as effective amount of a compound of formula (I) as defined in claim to patient in need thereof.

9. The compound as claimed in claim 1, wherein said pharmaceutical acceptable salt is selected from hydrochloride, hydrobromide, potassium or magnesium salt.
